(19) United States
(12) Patent Application Publication
(10) Pub. No.: US 2024/0207590 A1
(43) Pub. Date: Jun. 27, 2024

(54) MICRONEEDLE PATCH AND MANUFACTURING METHOD THEREOF

(71) Applicants: POSTECH Research and Business Development Foundation, Pohang-Si (KR); SNTEL Co., LTD, Pohang-Si (KR)

(72) Inventors: Geunbae LIM, Gyeongju-si (KR); Jungho LEE, Pohang-si (KR)

(73) Assignees: POSTECH Research and Business Development Foundation, Pohang-Si (KR); SNTEL Co., LTD, Pohang-Si (KR)

(21) Appl. No.: 18/543,180

(22) Filed: Dec. 18, 2023

(30) Foreign Application Priority Data
Dec. 23, 2022 (KR) 10-2022-0183012
Dec. 1, 2023 (KR) 10-2023-0172370

(51) Int. Cl. A61M 37/00

(52) U.S. Cl. CPC A61M 37/0015; A61M 2037/0023; A61M 2037/0053

(57) ABSTRACT

A microneedle patch according to one embodiment of the present disclosure includes: a base including an invasive part and a non-invasive part; and a microneedle located in the invasive part of the base, wherein at least a portion of the microneedle is loaded with drug, and no microneedle is located in the non-invasive part.

### CROSS-REFERENCE TO RELATED APPLICATIONS

The present application claims the benefit of priority to Korean Patent Application No. 10-2022-0183012, filed on Dec. 23, 2022, and Korean Patent Application No. 10-2023-0172370, filed on Dec. 1, 2023, in the Korean Intellectual Property Office. The disclosures of the above-listed application are hereby incorporated by reference herein in their entirety.

### TECHNICAL FIELD

The present disclosure relates to a microneedle patch and a method of manufacturing the same, and particularly to a microneedle patch including an invasive part where microneedles are located and a non-invasive part where no microneedle is located, and a method of manufacturing the microneedle patch.

### BACKGROUND

Drug injection using a conventional syringe has problems such as requiring injection by an expert and causing pain. To solve these problems, a microneedle-type drug injection system has been developed.

Microneedles are a system that delivers active ingredients into the skin by passing through the stratum corneum, the skin barrier layer. This new system combines the efficacy of the conventional syringe with the convenience of a patch, and technological advances have been made in recent years.

Microneedles pierce the stratum corneum of the skin and penetrate into the epidermis or dermis of the skin, where they remain in the skin for several minutes to several hours and are decomposed by body fluids to allow the drug to be absorbed into the body.

Generally, microneedles are used for delivery of active substances such as drugs and vaccines in vivo, detection of analytes in the body, and biopsy. Delivery of pharmaceutical or chemical active ingredients using microneedles is aimed at delivering active substances through the skin rather than through the biological circulatory system such as blood vessels or lymphatic vessels. Therefore, the microneedle needs to have sufficient physical strength to penetrate the skin, and it is desirable that it cause little pain.

The microneedle may be configured to load drugs at the pointed end and have a hollow central part. If the microneedle penetrate directly into the affected area, it may cause bleeding and inflammation.

### SUMMARY

In view of the above, the present disclosure provides a microneedle patch that minimizes bleeding and inflammation and a method of manufacturing the same.

A microneedle patch according to one embodiment of the present disclosure includes: a base including an invasive part and a non-invasive part; and a microneedle located in the invasive part of the base, wherein at least a portion of the microneedle is loaded with drug, and no microneedle is located in the non-invasive part.

In one aspect, the non-invasive part may be positioned in a central portion of the base, and the invasion part may be positioned in a puerperal portion of the base.

In one aspect, the invasive part may be positioned to surround the non-invasive part.

In one aspect, the microneedle may include a first portion and a second portion, and only the second portion may be loaded with the drug.

In one aspect, the microneedle may include a first portion and a second portion, and the second portion may be loaded with a higher concentration of the drug than the first portion.

In one aspect, the invasive part may be provided with a plurality of the microneedles, and a drug loading state of each microneedle may be determined by a distance from the non-invasive part.

In one aspect, the base may contain a biodegradable polymer.

In one aspect, the base may contain one or more of hyaluronic acid, carboxymethylcellulose, polyvinyl alcohol, chitosan, collagen, and polyvinyl pyrrolidone.

In one aspect, the base and at least a portion of the microneedle may contain a same material.

In one aspect, the drug loaded into the microneedle may include a drug having at least one of hemostatic effect and analgesic effect.

In one aspect, the drug loaded into the microneedle may include at least one of tranexamic acid, fibrin, thrombin, tannic acid, chitin, lidocaine, salicylic acid, ketoprofen, loxoprofen, flurbiprofen, piroxicam, felbinac, diclofenac diethylammonium, indomethacin, and antihistamine.

In one aspect, a height of the microneedle may be 100 μm to 5000 μm.

In one aspect, a radius of the microneedle may be 50 μm to 500 μm.

A method of manufacturing a microneedle patch, according to one embodiment of the present disclosure, includes: preparing a mold including an invasive section in which a groove is formed and a non-invasive section in which no groove is formed; and forming a microneedle patch including a microneedle and a base by applying a polymer onto the mold, wherein the microneedle patch includes an invasive part where the microneedle is located and a non-invasive part where no microneedle is located.

In one aspect, the non-invasive part may be positioned in a central portion of the manufactured microneedle patch, and the invasion part may be positioned in a peripheral portion of the microneedle patch.

In one aspect, the invasive part of the manufactured microneedle patch may be positioned to surround the non-invasive part of the microneedle patch.

In one aspect, the forming of the microneedle patch may include: forming a second portion of the microneedle by applying a polymer containing a drug to the groove of the mold; and forming a first portion of the microneedle and the base by applying a polymer onto the mold.

A method of manufacturing a microneedle patch, according to one embodiment of the present disclosure, includes: preparing a mold in which grooves are formed; forming a base including an invasive part having microneedles by applying a polymer onto the mold; and forming a non-invasive part by removing some of the microneedles.

In one aspect, the non-invasive part may be positioned in a central portion of the manufactured microneedle patch, and the invasion part may be positioned in a peripheral portion of the microneedle patch.

In one aspect, the forming of the base including the invasive part having the microneedles may include: forming second portions of the microneedles by applying a polymer containing a drug to the grooves of the mold; and forming first portions of the microneedles and the base by applying a polymer onto the mold.

The disclosed technology can have the following effects. However, it does not mean that a specific embodiment should include all of the following effects or only the following effects, and the scope of the disclosed technology should not be understood as being limited thereby.

The microneedle patch according to one embodiment of the present disclosure described above includes the invasive portion where the microneedle is located and the non-invasive part where the microneedle is not located, thereby preventing additional bleeding in the affected area and minimizing the inflammatory response.

In additionally, the microneedle patch can be manufactured in a simple and economical manner.

### BRIEF DESCRIPTION OF THE DRAWINGS

FIG. 1 shows a microneedle patch according to the present embodiment.

FIG. 2A shows configurations using the microneedle patch according to the present embodiment for hemostasis.

FIG. 2B shows an image of applying a microneedle patch with microneedles located in the entire area.

FIG. 2C shows an image of applying a microneedle patch including the invasive part and the non-invasive part to be located in the affected area.

FIGS. 3A and 3B show a cross-section and an actual image of a microneedle patch according to one embodiment, respectively.

FIG. 4 shows a method of manufacturing a microneedle patch according to one embodiment.

FIG. 5 shows a method of manufacturing a microneedle patch according to another embodiment.

FIG. 6 shows precipitation according to the content of tranexamic acid (TXA) loaded in ionized water.

FIGS. 7A to 7G show an image of the microneedle patch manufactured according to the present embodiment and an image of the patch actually applied to the affected area.

FIGS. 8A to 8D show the force applied to one microneedle (FIGS. 8A and 8B) and the force required to attach the patch (FIGS. 8C and 8D) for the microneedle patch.

FIGS. 9A and 9B show the results of rat liver hemostasis experiments with various substances.

FIG. 10A shows inflammation areas when various types of patches are attached to a liver wound.

FIG. 10B shows inflammation areas of FIG. 10A quantitatively.

FIGS. 11A and 11B show the results of the same experiment as FIGS. 9A and 9B on heparin-treated rats.

FIG. 12 shows an example of using the microneedle patch.

FIG. 13 shows the maximum adhesive force of the microneedle patch according to the present embodiment.

FIG. 14 shows a drug release profile of the microneedle patch according to the present embodiment.

FIG. 15 shows a measurement of the hemostatic performance of the microneedle patch according to the present embodiment on a skin wound of a rat.

FIG. 16 shows a measurement of the hemostatic performance of the microneedle patch according to the present embodiment on a heart wound of a rat.

FIG. 17 shows a measurement of the hemostatic performance of the microneedle patch according to the present embodiment on a kidney wound of a rat.

FIGS. 18A to 18C show a comparison of the inflammatory responses of a microneedle patch (P-MN patch) with invasive and non-invasive parts separated, a microneedle patch (MN patch) in which microneedles are located uniformly without distinction between invasive and non-invasive parts, and a patch containing no needle (Patch).

FIGS. 19A and 19B show a comparison of the inflammatory responses of a microneedle patch (P-MN patch) with invasive and non-invasive parts separated, a microneedle patch (MN patch) in which microneedles are located uniformly without distinction between invasive and non-invasive parts, and a patch containing no needle (Patch).

### DETAILED DESCRIPTION

The present disclosure may be changed in various ways and may have various embodiments, and specific embodiments are illustrated in the drawings and described in detail in the detailed description.

However, it should be understood that it is not intended to limit the present disclosure to the specific embodiments and that the present disclosure includes all changes, equivalents and substitutions which fall within the spirit and technological scope of the present disclosure.

Terms, such as first, second, etc., may be used to describe various components, but the components should not be restricted by the terms. These terms are used to only distinguish one component from another component. For example, a first component may be named a second component without departing from the scope of the present disclosure, or vice versa. The term “and/or” includes a combination of a plurality of related items or any one of the plurality of related items.

When it is described that a component is “coupled” or “connected” to another component, it should be understood that they may be directly coupled or connected to each other, but that other component may exist between them. On the other hand, when it is mentioned that a component is “directly coupled” or “directly connected” to another component, it should be understood that there are no other component between them.

The terms in the present specification are used to only describe specific embodiments and are not intended to limit the present disclosure. Singular expressions should be construed as including plural expressions unless clearly defined otherwise in the context. It is to be understood that in the present specification, a term, such as “include (or comprise)” or “have”, is intended to indicate that features, numbers, steps, operations, components, parts, or a combination thereof which are described in the present specification are present and does not exclude the possibility of the existence or addition of one or more other features, numbers, steps, operations, components, parts, or a combination thereof in advance.

Unless otherwise defined, all terms used herein, including technical or scientific terms, have the same meaning as commonly understood by a person of ordinary skill in the technical field to which the present disclosure pertains. Terms defined in commonly used dictionaries should be interpreted as having a meaning consistent with their contextual meaning in the relevant art, and should not be interpreted in an ideal or excessively formal sense unless explicitly defined in the present specification.

Hereinafter, preferred embodiments of the present disclosure will be described in detail with reference to the accompanying drawings. In describing the present disclosure, in order to facilitate overall understanding, like reference numerals are used for like components in the drawings, and redundant descriptions of the same components are omitted.

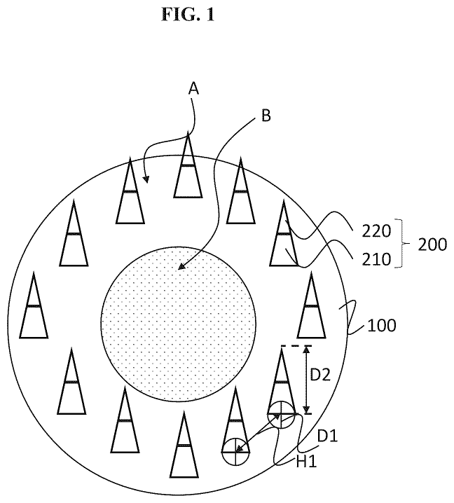

FIG. 1 shows a microneedle patch according to the present embodiment. Referring to FIG. 1, the microneedle patch according to the present embodiment includes a base 100 and microneedles 200 located on the base 100. According to one aspect, at least a portion of the microneedle 200 may be loaded with a drug.

The base 100 may contain a polymer material. For example, the base 100 may contain hyaluronic acid, but is not limited thereto. The base 100 may contain a biodegradable polymer, and specifically may contain one or more of hyaluronic acid, carboxymethylcellulose, polyvinyl alcohol, chitosan, collagen, and polyvinyl pyrrolidone.

The microneedle 200 may include a first portion 210 and a second portion 220. The second portion 220 may be located further away from the base 100 than the first portion 210. According to one aspect, the second portion 220 of the microneedle 200 may contain a drug therein, for example, a hemostatic agent. For example, the second portion 220 may contain a drug that has at least one of hemostatic effect and analgesic effect. In one embodiment, the second portion 220 may contain, but is not limited to, tranexamic acid. Specifically, the second portion 220 may contain a drug having hemostatic or analgesic effect, such as tranexamic acid, fibrin, thrombin, tannic acid, chitin, lidocaine, salicylic acid, ketoprofen, loxoprofen, flurbiprofen, piroxicam, felbinac, diclofenac diethylammonium, indomethacin, antihistamine, and the like. According to one aspect, the drug may be loaded only into the second portion of the microneedle.

According to one aspect, the base 100 and at least a portion of the microneedle 200 may contain the same material. More specifically, but not exclusively, the base 100 and the first portion 210 of the microneedle 200 may be connected to each other and may be made of the same material. For example, the base 100 and the first portion 210 of the microneedle 200 may contain hyaluronic acid. Depending on embodiments, the second portion of the microneedle may be loaded with a higher concentration of drug than the first portion. For example, the base 100 and the first portion 210 of the microneedle 200 may contain a low concentration of tranexamic acid. The second portion 220 of the microneedle 200 may contain hyaluronic acid containing a higher concentration of tranexamic acid than the first portion 210. Alternatively, the base 100 and the first portion 210 of the microneedle 200 may not contain tranexamic acid.

As shown in FIG. 1, the microneedle patch according to the present embodiment includes an invasive part A where the microneedle 200 is located and a non-invasive part B where no microneedle is located. The microneedle patch according to the present embodiment can deliver the drug through the invasive part A while the non-invasive part B is placed at an area where bleeding or severe wound occurs. Thus, the microneedle 200 does not invade the area where bleeding or severe wound is occurring, thereby preventing additional bleeding at the wound site and minimizing the inflammatory response.

As shown in FIG. 1, the non-invasive part B may be surrounded by the invasive part A. That is, the non-invasive part B may be positioned in a central portion of the patch and the invasive part A may be positioned in a peripheral portion. Therefore, when applying the patch, the non-invasive part B is located on the affected area and the invasive part A is located around the affected area, so that the drug can be delivered to the affected area by the microneedles around the affected area. However, the shapes of the invasive part A and the non-invasive part B may vary and are not limited to the shapes shown in FIG. 1. As shown in FIG. 1, the patch may be circular, but the shape of the patch may vary without being limited thereto.

Meanwhile, according to one aspect, the invasive part may be provided with a plurality of microneedles, and a drug loading state of each of the plurality of microneedles may be determined by a distance from the non-invasive part. For example, among the plurality of microneedles disposed in the invasive part, microneedles closer to the non-invasive part (i.e., affected area) may contain the drug, while microneedles farther from the non-invasive part may not contain the drug. In other words, when the patch is viewed transversely, the patch may be configured to include the non-invasive part (center), the invasive part containing the drug, and the invasive part (edge) containing no drug, which are arranged sequentially. Alternatively, the concentration of the drug contained in the microneedle in the invasive part may be adjusted depending on the distance from the wound, that is, the distance from the non-invasive part of the patch. For example, the patch may be configured such that the microneedle closer to the non-invasive part is loaded with a higher concentration of drug. Accordingly, it is possible to improve the strength of the entire microneedle patch while performing more efficient hemostasis or drug delivery to the affected area.

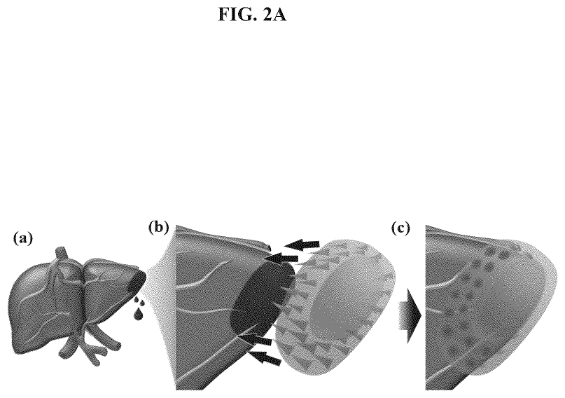

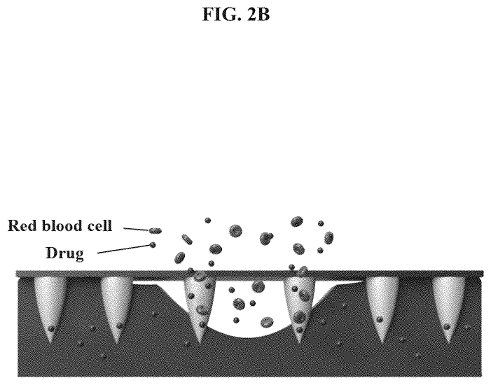

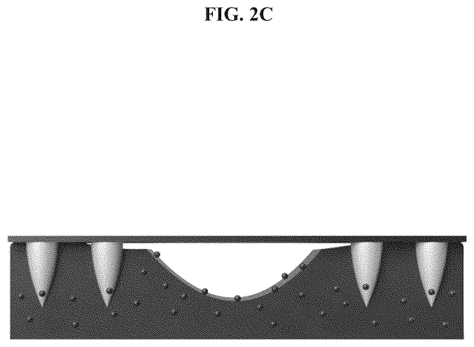

FIG. 2A shows configurations using the microneedle patch according to the present embodiment for hemostasis. (a) of FIG. 2A schematically shows a bleeding liver, and as shown in (b) of FIG. 2A and (c) of FIG. 2A, the microneedle patch according to the present embodiment may be attached. As described above, the microneedle patch according to the present embodiment may include the invasive part where the microneedle is located and the non-invasive part. As shown in (b) of FIG. 2A and (c) of FIG. 2A, the non-invasive part of the microneedle patch may be located in the affected area where bleeding occurs. In this case, because the microneedles of the microneedle patch do not invade the affected area, additional bleeding can be prevented and inflammation can be prevented.

FIG. 2B is an image of applying a microneedle patch with microneedles located in the entire area, and FIG. 2C is an image of applying a microneedle patch including the invasive part and the non-invasive part to be located in the affected area. Referring to FIG. 2B, in the case of the microneedle patch whose microneedles are located in the entire area, the microneedles are located even in the affected area where bleeding occurs, which may stimulate the affected area and cause additional bleeding. However, referring to FIG. 2C, the microneedle patch whose microneedles are located only in a certain area can prevent additional bleeding because the microneedles do not penetrate into the affected area. The drug can be delivered to the affected area through the invasive part where the microneedles are located to stop bleeding or treat the wound.

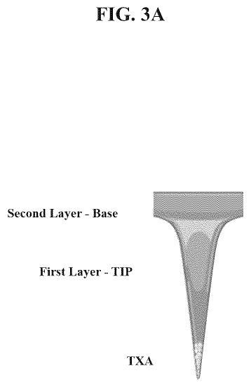

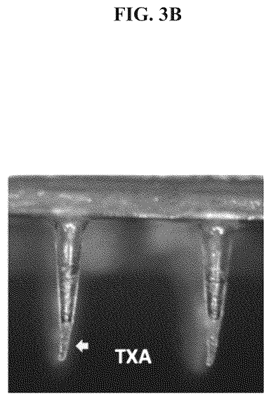

FIGS. 3A and 3B show a cross-section and an actual image of a microneedle patch according to one embodiment, respectively. The microneedle patch shown in FIGS. 3A and 3B is loaded with tranexamic acid (TXA) in the second portion. The base and needle shown in FIGS. 3A and 3B may include biodegradable polymers. FIG. 3B shows that the microneedles of the microneedle patch are loaded with TXA.

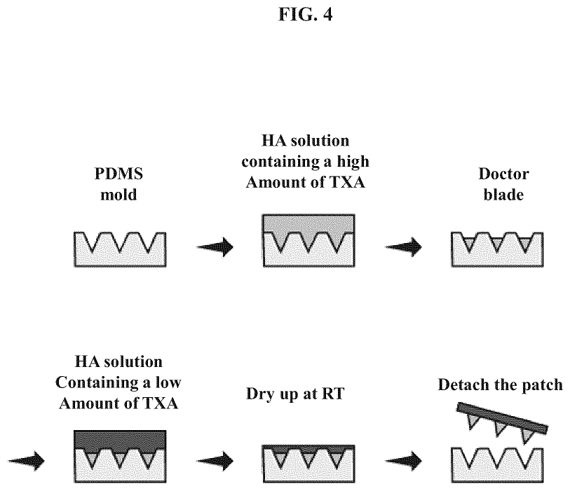

FIG. 4 shows a method of manufacturing a microneedle patch according to one embodiment. Referring to FIG. 4, after preparing a PDMS mold, a hyaluronic acid solution containing a high concentration of TXA may be applied. Through this, the tip portion of the microneedle loaded with the drug (the second portion 220 of the microneedle 200 shown in FIG. 1) is formed. Next, the first portion 210 and the base 100 of the microneedle 200 shown in FIG. 1 are formed using a hyaluronic acid solution containing a low concentration of TXA. After drying at room temperature, the microneedle patch is separated from the mold. Although not shown in FIG. 4, a step of removing the microneedle may be further included to form a non-invasive part corresponding to the affected area. In this case, removal of the microneedles may be accomplished by a physical method. When manufactured using this manner, the microneedle patch may be used by appropriately removing the microneedles depending on the shape of the affected area.

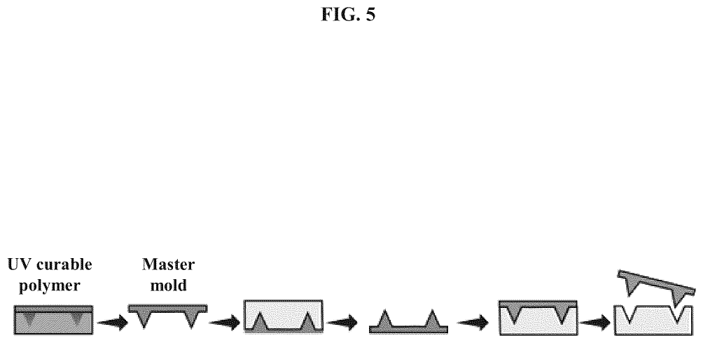

Alternatively, the microneedle patch according to the present embodiment may be manufactured using a mold with separate invasive and non-invasive sections. FIG. 5 shows a method of manufacturing a microneedle patch according to another embodiment. Referring to FIG. 5, a master mold is manufactured using UV curable polymer. In this case, the master mold includes an invasive section where the microneedles are located and a non-invasive section where no microneedle is located. Next, a microneedle patch loaded with a drug may be manufactured through a process of forming a mold using the master mold, applying hyaluronic acid containing the drug to the mold, and then separating it. As shown in FIG. 5, in the manufacturing method according to this embodiment, the microneedle patch is manufactured using the mold with the invasive section and the non-invasive section separated, and the step of removing the microneedle as in the manufacturing method of FIG. 4 may not be included.

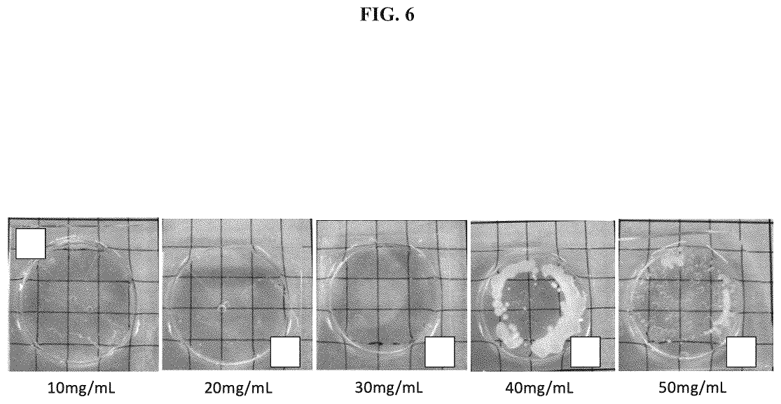

In one embodiment, the concentration of the drug loaded into the second portion 220 of the microneedle 200 of FIG. 1 may be 10 mg/mL to 30 mg/mL. This is the concentration range in which the drug can be uniformly distributed without precipitation occurring. FIG. 6 shows precipitation according to the content of TXA loaded in ionized water. Referring to FIG. 6, it can be seen that when the concentration of TXA is 10 mg/mL to 30 mg/mL, a uniform solid layer is formed, but when the concentration of TXA is greater than or equal to 40 mg/mL, precipitation occurs and the distribution of TXA in the patch becomes uneven.

Referring to FIG. 1, a radius D1 of the microneedle 200 may be 50 μm to 500 μm. In addition, a distance H1 between the microneedles 200 may be 100 μm to 5000 μm. Further, a height D2 of the microneedle 200 may be 100 μm to 5000 μm. When the height of the microneedle is less than 100 μm, it is difficult to sufficiently pierce the stratum corneum of the skin, and when it is larger than 5000 μm, it is structurally unstable due to the high aspect ratio, and tends to break without penetrating the skin. A tip diameter of the microneedle 200 may be 5 μm to 50 μm. The tip diameter of the microneedle 200 refers to a hemispherical structure observed at the tip of the microneedle. A tip angle of the microneedle 200 refers to an angle of the tip of the microneedle observed from the side, and refers to an acute angle measured based on an imaginary vertex, such as when the tip of the microneedle has an ideal cone structure. The tip diameter and the tip angle of the microneedle are designed to be sharp enough to penetrate the skin.

That is, the microneedle patch according to the present embodiment can minimize invasion because the size of the microneedle 200 is small.

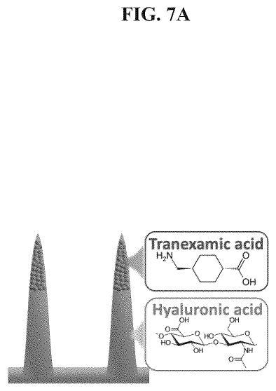

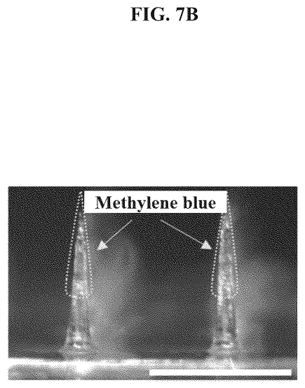

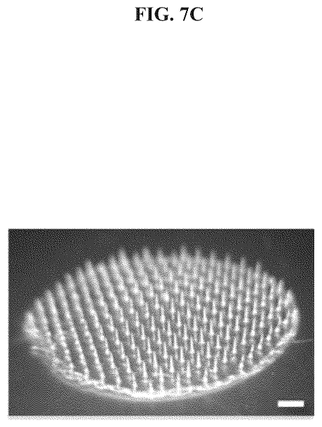

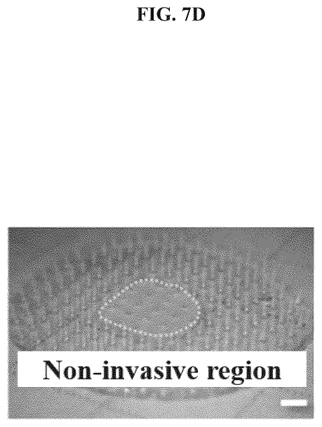

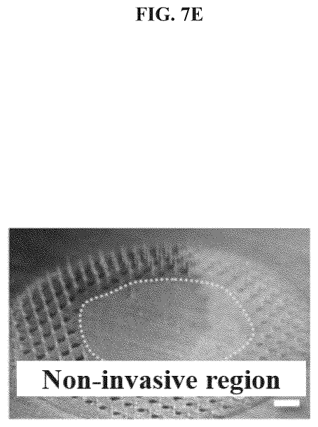

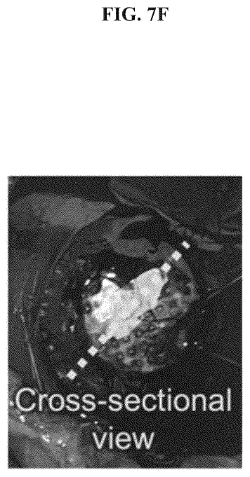

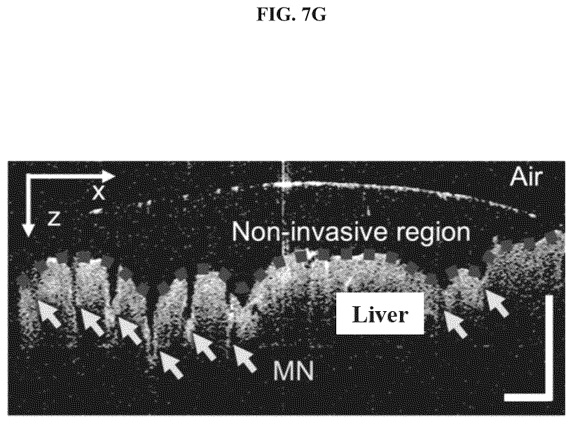

FIGS. 7A to 7G show an image of the microneedle patch manufactured according to the present embodiment and an image of the patch actually applied to the affected area. Referring to FIG. 7A, the microneedle patch contains hyaluronic acid, and one end of the microneedle is loaded with TXA. FIG. 7B shows an image of the actually manufactured microneedle patch, and the presence of TXA was confirmed by loading methylene blue. As shown in FIG. 7B, the portion loaded with TXA was confirmed to be blue. FIG. 7C shows an image of a microneedle patch with microneedles formed uniformly without distinction between invasive and non-invasive parts, and FIGS. 7D and 7E show images of microneedle patches with separate invasive and non-invasive parts. As shown in FIG. 7F, part of the liver tissue of a male rat was removed, and then a microneedle patch with invasive and non-invasive parts was attached according to the present embodiment. FIG. 7G shows an image of the interface of the liver tissue to which the microneedle patch is attached. As shown in FIG. 7G, it can be seen that the microneedles were not located in the affected area (non-invasive region), but that the microneedles (MN) were located only in the area surrounding the affected area.

Since the force required for attachment of the microneedle patch, in which microneedles are located only in certain areas, is small, the patch can be attached to the affected area with minimal pressure.

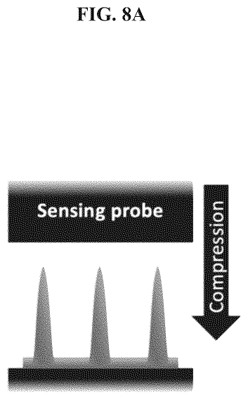

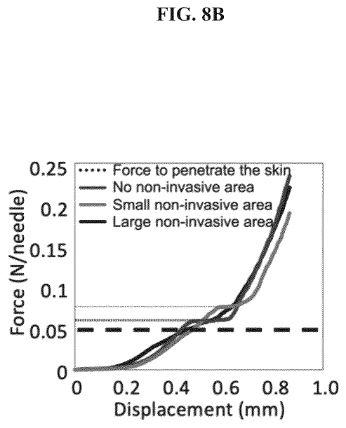

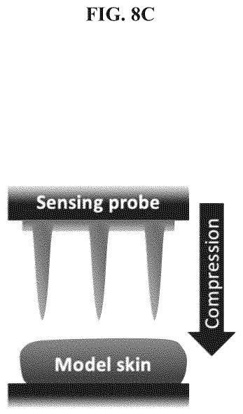

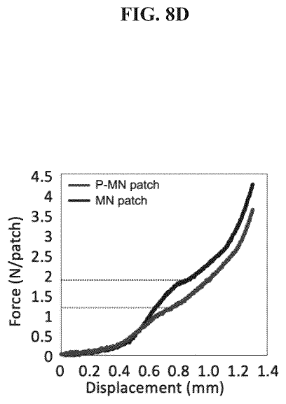

FIGS. 8A to 8D show the force applied to one microneedle (FIGS. 8A and 8B) and the force required to attach the patch (FIGS. 8C and 8D) for the microneedle patch. Referring to FIGS. 8A and 8B, it can be seen that the force applied to one microneedle is similar for a microneedle patch without the non-invasive part (No non-invasive area) and microneedle patches with the non-invasive part (Small non-invasive area and Large non-invasive area). On the other hand, referring to FIGS. 8C and 8D, it can be seen that a microneedle patch (P-MN patch) in which the invasive and non-invasive parts are separated can be attached with a lower force than a microneedle patch (MN patch) in which the invasive and non-invasive parts are not distinguished and the microneedles are positioned uniformly. That is, the microneedle patch according to the present embodiment can reduce the pressure for attachment, thereby reducing the effect of pressure on the bleeding wound.

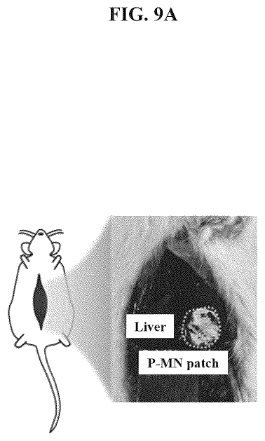

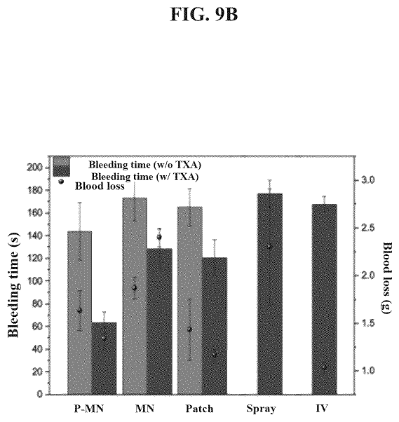

FIGS. 9A and 9B show the results of rat liver hemostasis experiments with various substances. As shown in FIG. 9A, liver wound was induced in experimental rats. Then, bleeding amount and hemostasis time were measured for a microneedle patch with separate invasive and non-invasive parts (P-MN), a microneedle patch with microneedles positioned uniformly without distinction between invasive and non-invasive parts (MN), and a patch containing no needle (Patch), a spray (Spray), and an intravenous injection (IV), and the results are shown in FIG. 9B. Referring to FIG. 9B, it can be seen that the microneedle patch (P-MN) with separate invasive and non-invasive parts reduced bleeding time the most. On the other hand, the microneedle patch (MN), in which microneedles are positioned uniformly without distinction between invasive and non-invasive parts, increased bleeding. Through this, it has been confirmed that the microneedles of the microneedle patch may cause additional bleeding if the microneedles are placed in the affected area.

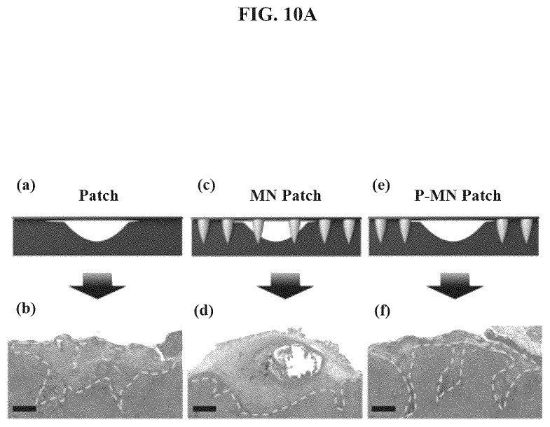

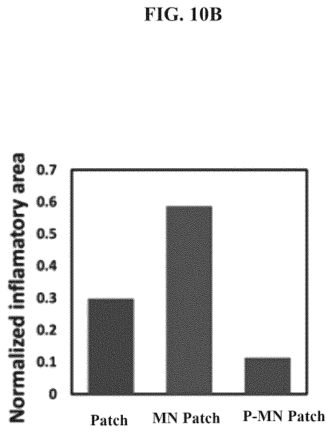

FIG. 10A shows inflammation areas when various types of patches are attached to a liver wound, and FIG. 10B shows inflammation areas of FIG. 10A quantitatively. That is, (a) of FIG. 10A shows a patch including no microneedle (Patch), and (b) of FIG. 10A shows the location of the inflammation area when the patch is attached to the liver wound. (c) of FIG. 10A shows a microneedle patch (MN patch) in which microneedles are positioned uniformly without distinction between invasive and non-invasive parts, and (d) of FIG. 10A shows the location of the inflammation area when the patch is attached to the liver wound. (e) of FIG. 10A shows a microneedle patch (P-MN patch) with invasive and non-invasive parts separated, and (f) of FIG. 10A shows the location of the inflammation area when the patch is attached to the liver wound.

In addition, FIG. 10B shows the inflammatory areas in (b) of FIG. 10A, (d) of FIG. 10A and (f) of FIG. 10A quantitatively. Referring to FIGS. 10A to 10B, it can be seen that the inflammation area is the largest and the inflammation area level is also the highest in the microneedle patch (MN patch) where the microneedles are located uniformly without distinction between invasive and non-invasive parts.

Referring to (f) of FIG. 10A, it can be seen that in the case of the microneedle patch (P-MN patch) with the invasive and non-invasive parts separated, the inflammation area appears narrower in the part where no microneedle is located, and the inflammatory response appears weaker compared to the microneedle patch (MN patch) where the microneedles are located uniformly as shown in FIG. 10B. In addition, it can be seen that the inflammation area level of the microneedle patch (P-MN patch) where the invasive and non-invasive parts are separated, was the lowest.

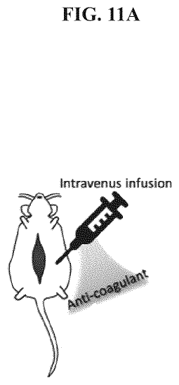

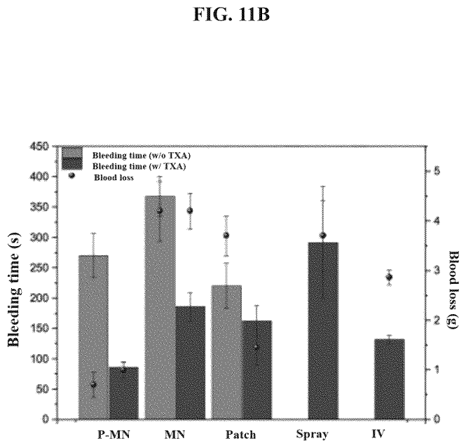

In the medical field, heparin, as an anticoagulant, is commonly administered to patients. Accordingly, a hemostasis experiment was performed on the liver wound of heparin-treated rats in the same manner as in FIGS. 9A and 9B and is shown in FIGS. 11A and 11B. FIG. 11A schematically shows the heparin-treated rat, and bleeding amount and hemostasis time were measured for a microneedle patch with separate invasive and non-invasive parts (P-MN), a microneedle patch with microneedles positioned uniformly without distinction between invasive and non-invasive parts (MN), and a patch containing no needle (Patch), a spray (Spray), and an intravenous injection (IV), and the results are shown in FIG. 11B. The results in FIG. 11B are similar to the results in FIG. 9B. In other words, it was confirmed that the microneedle patch (P-MN) with separate invasive and non-invasive parts reduced bleeding time the most. However, in the case of FIG. 11B, since the self-coagulation function was reduced due to the injection of heparin, the amount of bleeding was found to slightly increase due to the attachment of the microneedle patch.

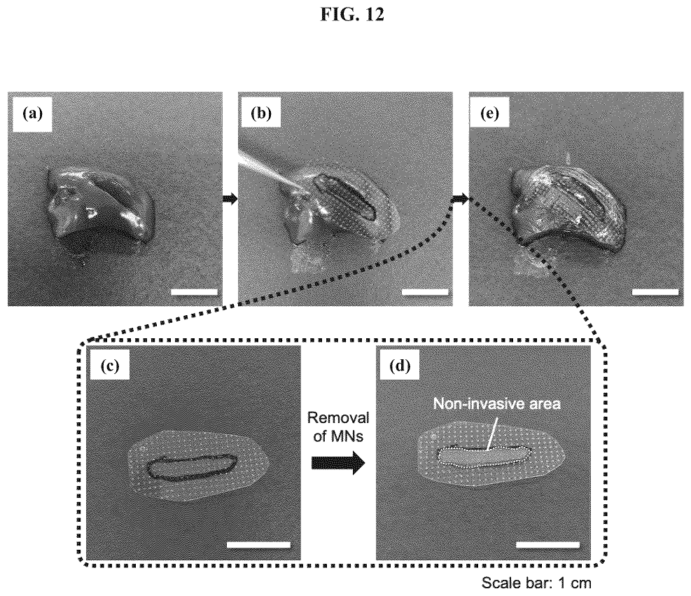

The microneedle patch according to the present embodiment may be used by appropriately removing the microneedles depending on the usage environment. FIG. 12 shows an example of using the microneedle patch. Referring to (a) of FIG. 12, a liver with an asymmetric lesion is shown. As shown in (b) of FIG. 12, after placing the microneedle patch on the liver, the area where the non-invasive part is to be located is indicated based on the shape of the wound. Next, as shown in (c) of FIGS. 12 and (d) of FIG. 12, the microneedles located in the non-invasive part may be removed, and the microneedle patch may be attached to the asymmetrical affected area as shown in (e) of FIG. 12. That is, the microneedle patch according to the present embodiment can be used by removing the microneedles depending on the usage environment. Therefore, it can be applied to affected areas of various shapes.

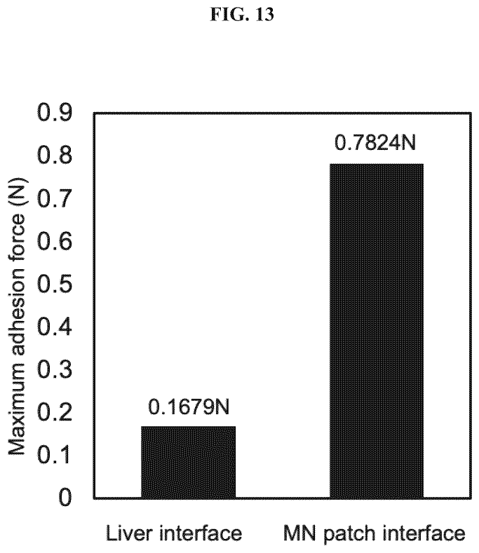

FIG. 13 shows the maximum adhesion force of the microneedle patch according to the present embodiment. FIG. 13 shows the maximum adhesion force between liver tissue and liver tissue (Liver interface) and the maximum adhesion force between liver tissue and microneedle patch (MN patch interface). Referring to FIG. 13, the maximum adhesion force between the liver tissue and the microneedle patch (MN patch interface) was higher than the maximum adhesion force between the liver tissue and the liver tissue (Liver interface), and therefore, it can be seen that the micro needle patch according to the present embodiment is adhered stably to the liver tissue.

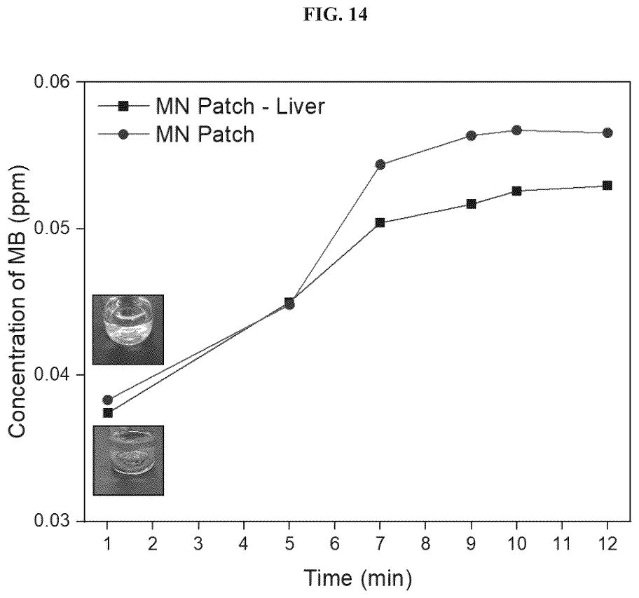

FIG. 14 shows a drug release profile of the microneedle patch according to the present embodiment. In FIG. 14, the broken line indicates the amount of drug dissolved over time when the microneedle is attached to the liver and then exposed to a mimic body fluid (phosphate buffered saline), and the solid line indicates the amount of drug dissolved over time when the microneedle is exposed to the mimic body fluid (phosphate buffered saline) without being attached to the liver. This shows that when the microneedle patch is attached to the tissue, the drug tends to be absorbed to a certain level into the skin even when there is a lot of body fluid around the tissue. From FIG. 14, it can be seen that the microneedle patch according to the present embodiment stably releases the drug into the liver tissue.

The microneedle patch according to the present embodiment has excellent hemostatic performance in various bleeding sites.

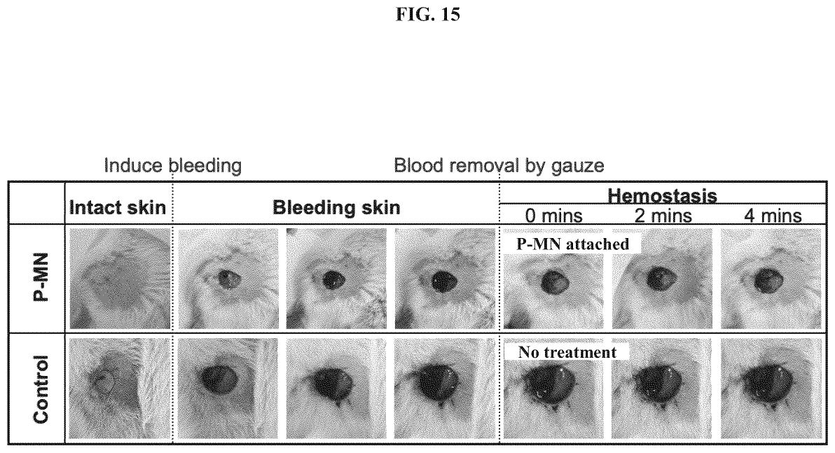

FIG. 15 shows a measurement of the hemostatic performance of the microneedle patch according to the present embodiment on a skin wound of a rat. Compared to the control, it can be seen that the microneedle patch (P-MN) according to the present embodiment has excellent hemostatic performance.

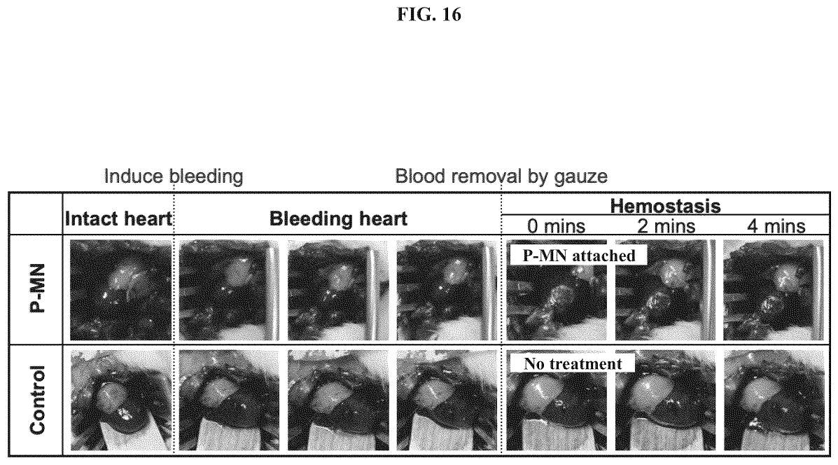

FIG. 16 shows a measurement of the hemostatic performance of the microneedle patch according to the present embodiment on a heart wound of a rat. Compared to the control, it can be seen that the microneedle patch (P-MN) according to the present embodiment has excellent hemostatic performance.

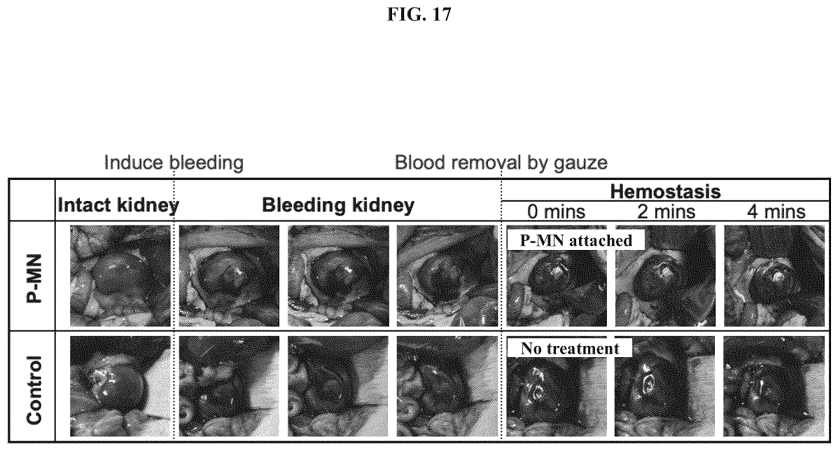

FIG. 17 shows a measurement of the hemostatic performance of the microneedle patch according to the present embodiment on a kidney wound of a rat. Compared to the control, it can be seen that the microneedle patch (P-MN) according to the present embodiment has excellent hemostatic performance.

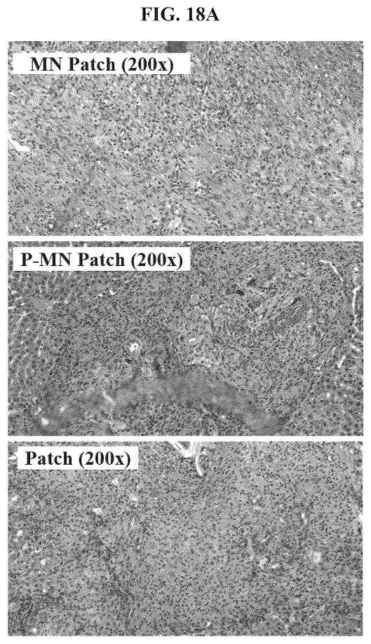

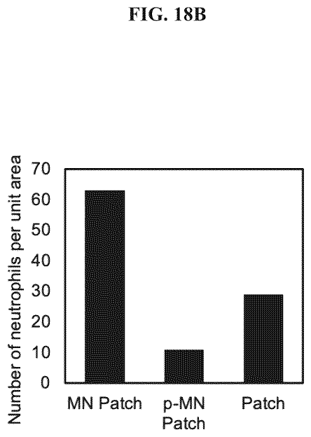

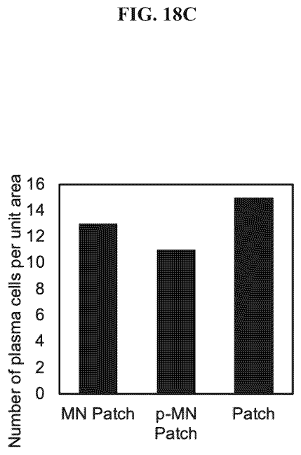

FIGS. 18A to 18C show a comparison of the inflammatory responses of a microneedle patch (P-MN patch) with invasive and non-invasive parts separated, a microneedle patch (MN patch) in which microneedles are located uniformly without distinction between invasive and non-invasive parts, and a patch containing no needle (Patch). FIG. 18A shows hematoxylin and eosin stained images of the microneedle patch (P-MN patch) with invasive and non-invasive parts separated, the microneedle patch (MN patch) in which microneedles are located uniformly without distinction between invasive and non-invasive parts, and the patch containing no needle (Patch), and FIG. 18B shows quantification of neutrophils per unit area for the microneedle patch (P-MN patch) with invasive and non-invasive parts separated, the microneedle patch (MN patch) in which microneedles are located uniformly without distinction between invasive and non-invasive parts, and the patch containing no needle (Patch). Next, FIG. 18C shows the quantification of plasma cells per unit area for the microneedle patch (P-MN patch) with invasive and non-invasive parts separated, the microneedle patch (MN patch) in which microneedles are located uniformly without distinction between invasive and non-invasive parts, and the patch containing no needle (Patch). Referring to FIGS. 18A to 18C, it can be seen that the microneedle patch (P-MN patch) with invasive and non-invasive parts separated has the lowest the number of neutrophils per unit area, indicating the least inflammatory response.

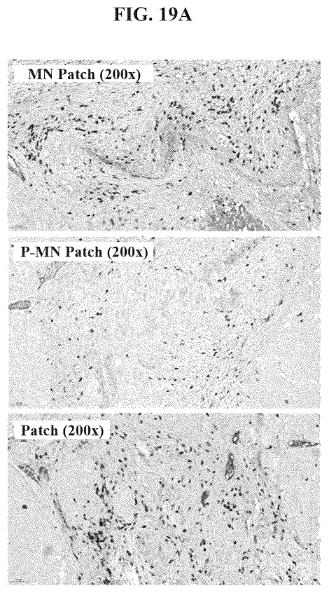

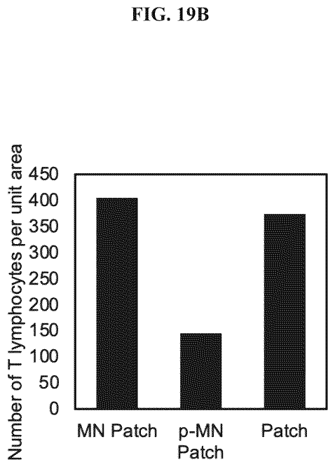

FIGS. 19A and 19B show a comparison of the inflammatory responses of a microneedle patch (P-MN patch) with invasive and non-invasive parts separated, a microneedle patch (MN patch) in which microneedles are located uniformly without distinction between invasive and non-invasive parts, and a patch containing no needle (Patch). FIG. 19A shows images of CD3 immunohistochemical staining of the microneedle patch (P-MN patch) with invasive and non-invasive parts separated, the microneedle patch (MN patch) in which microneedles are located uniformly without distinction between invasive and non-invasive parts, and the patch containing no needle (Patch), FIG. 19B shows quantification of the number of T lymphocytes per unit area for the microneedle patch (P-MN patch) with invasive and non-invasive parts separated, the microneedle patch (MN patch) in which microneedles are located uniformly without distinction between invasive and non-invasive parts, and the patch containing no needle (Patch).

Referring to FIGS. 19A and 19B, it can be seen that the microneedle patch (P-MN patch) with invasive and non-invasive parts separated has the lowest the number of T lymphocytes per unit area, indicating the least inflammatory response.

As described above, the microneedle patch according to the present embodiment includes the invasive part where the microneedles are located and the non-invasive part where no microneedle is located. Accordingly, the drug can be delivered without direct penetration of the microneedle into the affected area, and additional bleeding or inflammatory response can be minimized. The microneedle patch can be manufactured using a mold including an invasive section and a non-invasive section, or can be manufactured by physically removing microneedles, so the manufacturing method thereof is simple and the microneedle patch can be applied to affected areas of various shapes.

What is claimed is:

1. A microneedle patch comprising:
a base including an invasive part and a non-invasive part; and
a microneedle located in the invasive part of the base,
wherein at least a portion of the microneedle is loaded with drug, and
no microneedle is located in the non-invasive part.

2. The microneedle patch of claim 1, wherein the non-invasive part is positioned in a central portion of the base, and
the invasion part is positioned in a puerperal portion of the base.

3. The microneedle patch of claim 1, wherein the invasive part is positioned to surround the non-invasive part.

4. The microneedle patch of claim 1, wherein the microneedle includes a first portion and a second portion, and
only the second portion is loaded with the drug.

5. The microneedle patch of claim 1, wherein the microneedle includes a first portion and a second portion, and
the second portion is loaded with a higher concentration of the drug than the first portion.

6. The microneedle patch of claim 1, wherein the invasive part is provided with a plurality of the microneedles, and
a drug loading state of each microneedle is determined by a distance from the non-invasive part.

7. The microneedle patch of claim 1, wherein the base contains a biodegradable polymer.

8. The microneedle patch of claim 1, wherein the base contains one or more of hyaluronic acid, carboxymethylcellulose, polyvinyl alcohol, chitosan, collagen, and polyvinyl pyrrolidone.

9. The microneedle patch of claim 1, wherein the base and at least a portion of the microneedle contain a same material.

10. The microneedle patch of claim 1, wherein the drug loaded into the microneedle includes a drug having at least one of hemostatic effect and analgesic effect.

11. The microneedle patch of claim 1, wherein the drug loaded into the microneedle includes at least one of tranexamic acid, fibrin, thrombin, tannic acid, chitin, lidocaine, salicylic acid, ketoprofen, loxoprofen, flurbiprofen, piroxicam, felbinac, diclofenac diethylammonium, indomethacin, and antihistamine.

12. The microneedle patch of claim 1, wherein a height of the microneedle is 100 μm to 5000 μm.

13. The microneedle patch of claim 1, wherein a radius of the microneedle is 50 μm to 500 μm.

14. A method of manufacturing a microneedle patch, the method comprising:
preparing a mold including an invasive section in which a groove is formed and a non-invasive section in which no groove is formed; and
forming a microneedle patch including a microneedle and a base by applying a polymer onto the mold,
wherein the microneedle patch includes an invasive part where the microneedle is located and a non-invasive part where no microneedle is located.

15. The method of claim 14, wherein the non-invasive part is positioned in a central portion of the manufactured microneedle patch, and
the invasion part is positioned in a peripheral portion of the microneedle patch.

16. The method of claim 14, wherein the invasive part of the manufactured microneedle patch is positioned to surround the non-invasive part of the microneedle patch.

17. The method of claim 14, wherein the forming of the microneedle patch includes:
forming a second portion of the microneedle by applying a polymer containing a drug to the groove of the mold; and
forming a first portion of the microneedle and the base by applying a polymer onto the mold.

18. A method of manufacturing a microneedle patch, the method comprising:
preparing a mold in which grooves are formed;
forming a base including an invasive part having microneedles by applying a polymer onto the mold; and
forming a non-invasive part by removing some of the microneedles.

19. The method of claim 18, wherein the non-invasive part is positioned in a central portion of the manufactured microneedle patch, and
the invasion part is positioned in a peripheral portion of the microneedle patch.

20. The method of claim 18, wherein the forming of the base including the invasive part having the microneedles includes:
forming second portions of the microneedles by applying a polymer containing a drug to the grooves of the mold; and
forming first portions of the microneedles and the base by applying a polymer onto the mold.
